(19) United States
(12) Patent Application Publication
(10) Pub. No.: US 2022/0273484 A1
(43) Pub. Date: Sep. 1, 2022

(54) INTRAORAL FERRULE WITH ARCH FOR PREVENTING SNORING AND APNOEA

(71) Applicants: Miguel NAVARRO SEGURA, Novelda - Alicante (ES); Federico Juan Carlos MANGADA MARTINEZ, Alicante (ES)

(72) Inventor: Miguel NAVARRO SEGURA, Novelda Alicante (ES)

(73) Assignees: Miguel NAVARRO SEGURA, Novelda - Alicante (ES); Federico Juan Carlos MANGADA MARTINEZ, Alicante (ES)

(21) Appl. No.: 17/286,255

(22) PCT Filed: Oct. 22, 2019
(86) PCT No.: PCT/ES2019/070716

§ 371 Date: Apr. 16, 2021

(30) Foreign Application Priority Data
Oct. 24, 2018 (ES) U201831605

(51) Int. Cl. A61F 5/56

(52) U.S. Cl. CPC A61F 5/566

(57) ABSTRACT

An intraoral ferrule with arch for preventing snoring and apnea. The arch of which protrudes from the anterior inner part of the ferrule and with extensions to the posterior part that run parallel to the bite plane to a point at which they turn and are joined to a curved region, forming the end of the arch, which retains the tongue, thereby preventing the possible displacement thereof towards the posterior part of the user's buccal cavity.

### DESCRIPTION

### OBJECT OF THE INVENTION

The purpose of the present utility model is to present a new intraoral ferrule with a large arch which arises from the inner anterior region of the ferrule to the posterior part for preventing snoring and apnea, which, being placed in the user's maxilla, prevents the possible displacement of the tongue towards the posterior part of said user's buccal cavity, thereby preventing the possible obstruction of the airway due to the overlap of the posterior part of the tongue, or soft palate, on the posterior pharyngeal wall.

It has a special application in the sector of otorhinolaryngology, where there is a need to correct these problems.

### BACKGROUND OF THE INVENTION

Up until now, various devices are reported in the current state of the art for solving problems with snoring and apnea, with specific technical features for each case.

Document EP-A-0,312.368 comprises an intraoral device for preventing snoring. This device consists of a U-shaped mouthpiece which is shaped to the user's upper dental arching and includes an inclined lower ramp in order to interlock with the mandibular dentition. Normal movements of the mouth, such as closing the jaws, will cause part of the mandibular dentition to interlock with the lower part of the ramp, pushing the lower jaw forward by a cam in order to increase the separation between the base of the tongue and the posterior wall of the pharynx.

Said device is clearly different from the one described in the present Invention Patent.

Invention Patent ES 2217284 T3 describes a device for preventing stertorous breathing or snoring and adapted to be fastened in the upper part of a person's mouth, comprising a non-rigid and flexible transverse bridge for fastening in the upper part of a person's mouth, preferably in the rear part of the upper part of the mouth, and a member for grasping the tongue, which extends downward from the non-rigid and flexible transverse bridge.

The main difference with respect to said Invention Patent is that instead of grasping the tongue by default and in a fixed manner, in the present invention, a posterior arch is available, just so that in case of a possible displacement of the tongue, the movement thereof can be limited, and, therefore, it can be maintained in a position wherein the airway is not obstructed due to the overlap of the posterior part of the tongue, or soft palate, on the posterior pharyngeal wall.

It should also be commented that our invention is based on an arch which starts from the anterior inner region, not on a transverse bridge.

Patent ES 2118365 T3 defines a dental apparatus for the treatment of snoring episodes and obstructive sleep apnea. This apparatus, made to be worn by a patient when sleeping, has an upper member which is shaped to the patient's upper maxillary dentition, a lower member which is shaped to the patient's mandibular dentition, and a connector means for releasably coupling said upper and lower members to each other, wherein said connector means adjustably maintains said lower member in an anterior position, projected in relation to said upper member, and wherein said connector means enable the lateral movement of said lower member in relation to said upper member in said projected position.

This dental apparatus differs from the present utility model, since it is essentially based on positioning the upper member which is shaped to the patient's upper maxillary dentition, with respect to the lower member which is shaped to the patient's lower maxillary dentition by means of a connector which positions said members and, therefore, enables said positions to be adjusted to each other.

Utility Model U200800413 describes an intraoral device for preventing snoring and apnea. Said device is composed of a body, which fits into the user's maxilla, and exerts uniform pressure thereon, said body incorporating a fin in the posterior part thereof which can vary in shape and size according to the morphology of the user's mouth.

The differences with respect to the present invention are significant, due to the fact that it is a palate, which has a fin, which is stuck in a certain point of the tongue, causing long-term damage to it, in addition to making correct salivation difficult.

Although the dental apparatuses of the state of the art have proven effective for maintaining the jaw in a projected position in order to improve the evidence of the airway, they sometimes result in undesirable side effects. One of the most common side effects is damage to the temporomandibular joint and related jaw muscles and ligaments, especially in individuals who have a tendency to grind their teeth as they sleep. Damage to the temporomandibular joint has been associated with a wide variety of physical illnesses, including migraine headaches. Therefore, many individuals suffering from snoring and sleep apnea disorders are unable to tolerate existing anti-snoring dental apparatuses for long periods of time.

The closest background documents are European patent EP2380533 and utility model ES1070884, which have an arch in the posterior part of the ferrule protruding from the region of the molars at a downwards angle so that this posterior arch prevents the displacement of the tongue. However, in both inventions, the arch protrudes from the posterior part of the ferrule, which keeps the arch from having some flexibility and when swallowing saliva, this stiffness is very annoying for the user who rejects it or even, due to the force made by the tongue when swallowing saliva, the arch may break or the ferrule may become dislodged.

The aim of this novel intraoral ferrule with arch for preventing snoring and apnea is to prevent these drawbacks by giving the arch some flexibility since the arch protrudes from the inner and anterior part of the ferrule and goes parallel to the bite plane, separated a few mm from the bite plane so that when the action of swallowing saliva is done, it does not have any friction or interference when making the movement towards the palate in order to facilitate the action of swallowing.

At no time is an intraoral ferrule with an arch for preventing snoring and apnea which starts from the inner anterior region of the ferrule as described in the present invention patent reflected in the state of the art.

### DESCRIPTION OF THE INVENTION

In order to alleviate or, if appropriate, eliminate all the aforementioned drawbacks, this novel intraoral ferrule is presented with an arch which starts from the anterior inner region of the ferrule to the posterior part and goes parallel to the bite plane separated a few mm therefrom so that when the action of swallowing saliva is done, it does not have any friction or interference when making the movement towards the palate in order to facilitate the action of swallowing.

Due to the distance between the point from which the arch protrudes in the inner anterior part of the ferrule and the inclined posterior part, overall, the arch has a certain flexibility which enables it to offer less resistance to the tongue when swallowing saliva.

The more anterior the arch protrudes, the more distance there is with the posterior inclined region of the arch and the more flexibility and less resistance of the arch on the tongue are achieved when swallowing saliva.

The most important advantages of this novel intraoral ferrule are:
Lower risk of breakage of the arch due to the flexibility thereof.
Less rejection of the user by facilitating swallowing saliva by retaining the tongue less.

### DESCRIPTION OF THE DRAWINGS

As a complement to the present description, and for the purpose of helping to make the features of the invention more readily understandable, said description is accompanied by a series of figures constituting an integral part of the same, which by way of illustration and not limitation represent the following:

FIG. 1 shows a top view of the intraoral ferrule with arch starting from the anterior region to the posterior region.

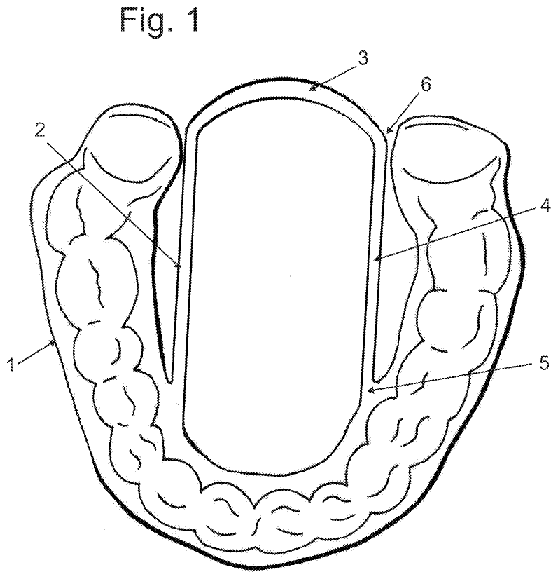

FIG. 2 shows a perspective view of the intraoral ferrule with arch for preventing snoring and apnea.

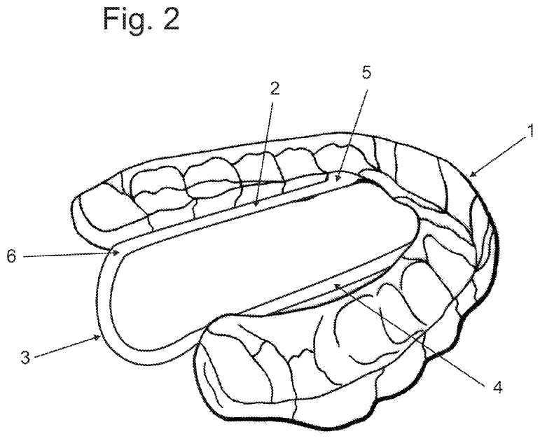

FIG. 3 shows a side cross-sectional view of an example of application of the intraoral ferrule with arch for preventing snoring and apnea, with the tongue in the normal resting state thereof.

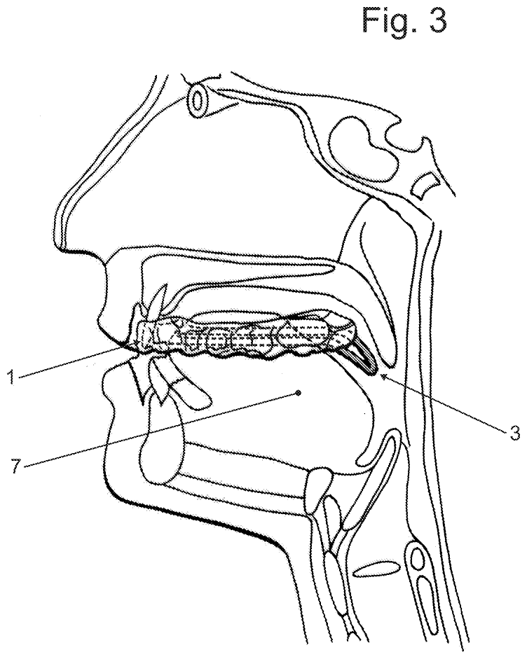

FIG. 4 shows a side view of the ferrule showing how the flexibility of the intermediate extensions of the arch enables a vertical displacement of the final curved region of the arch.

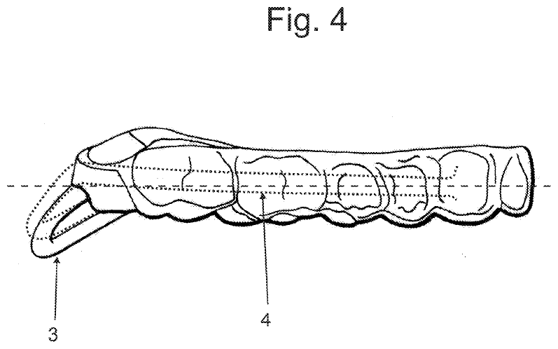

FIG. 5 shows a top view of the ferrule wherein it shows that the extensions protruding from the anterior inner part have a shape which is not straight.

### PREFERRED EMBODIMENT OF THE INVENTION

This intraoral ferrule with arch for preventing snoring and apnea is composed of a ferrule (1) which fits into the user's teeth, said ferrule incorporating an arch (2).

This arch (2) is U-shaped and protrudes from the anterior inner part (5) of the ferrule, at the height of the canines and the extensions (4) of which go parallel to the bite plane, separated a few mm from the plane thereof, to a point (6) at which they turn towards the lower part and are joined together by a curved region (3) that is the one which acts by retaining the tongue (7). The extensions may not go parallel to the bite plane and have a maximum inclination of 15° with respect to this plane towards the palate or towards the tongue.

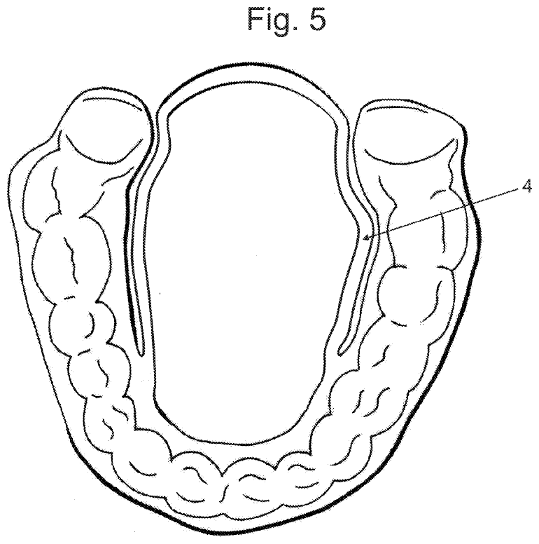

In another preferred embodiment, as seen in FIG. 5, the extensions (4) which go parallel to the bite plane, instead of being straight and parallel to each other, can have another varied shape as long as they are parallel to the bite plane and the flexibility thereof enables a displacement of the curved region (3) which forms the end of the arch with respect to the bite plane.

In other preferred embodiments, the arch (2) which protrudes from the anterior inner part (5) does so at the height of the incisors, the canines or the premolars. It must always be taken into account that the further back it protrudes, the less elasticity is conferred to the arch since these extensions are shorter.

Likewise, in other alternative embodiments, the region (3) which forms the end of the arch, and that is the one which acts by retaining the user's tongue (7), has any other geometric shape that does not have to be curved.

Once the nature of the present invention has been sufficiently described, it only remains to add that said invention may undergo certain variations in the composition thereof, as long as said alterations do not substantially vary the features claimed below.

1. An intraoral ferrule with arch for preventing snoring and apnea composed of a ferrule which fits into the user's teeth and which incorporates an arch wherein the arch is U-shaped and protrudes from the anterior inner part of the ferrule and the extensions of which go parallel to the bite plane to a point in the posterior final part of the ferrule at which they turn towards the lower part and are joined together by a region which forms the end of the arch and that is the one which acts by retaining the user's tongue.

2. The intraoral ferrule with arch for preventing snoring and apnea, according to claim 1, wherein the extensions which go parallel to the bite plane are flexible.

3. The intraoral ferrule with arch for preventing snoring and apnea, according to claim 1, wherein the arch which protrudes from the anterior inner part does so at the height of the incisors, the canines or the premolars.

4. The intraoral ferrule with arch for preventing snoring and apnea, according to claim 1, wherein the region which forms the end of the arch, and that is the one which acts by retaining the user's tongue, has a curved shape or any other geometric shape.

5. The intraoral ferrule with arch for preventing snoring and apnea, according to claim 1, wherein the extensions may not go parallel to the bite plane and have a maximum inclination of 15° with respect to this plane.

6. The intraoral ferrule according to claim 1, wherein
the extensions are flexible.

7. An intraoral ferrule comprising:
an arch for preventing snoring and apnea; and
a ferrule configured to fit into a user's teeth and which incorporates said arch, wherein
the arch is U-shaped, and
the arch protrudes from the anterior inner part of the ferrule and extensions od said arch are configured to go parallel to a bite plane to a point in the posterior final part of the ferrule.

8. The intraoral ferrule according to claim 7, wherein
the extensions are configured to be placed parallel to the bite plane are flexible.

9. The intraoral ferrule according to claim 6, wherein
the extensions are flexible.

10. The intraoral ferrule according to claim 7, wherein
the arch protrudes from the anterior inner part does so at the height of the incisors, the canines or the premolars.

11. The intraoral ferrule according to claim 7, wherein
the region has a curved shape.

12. The intraoral ferrule according to claim 7, wherein
the region has a geometric shape other than a curved shape.

13. The intraoral ferrule according to claim 7, wherein
the extensions may not go parallel to the bite plane and have a maximum inclination of 15° with respect to the bite plane.

14. The intraoral ferrule according to claim 7, wherein
the extensions have a maximum inclination of 15° with respect to the bite plane.

15. An intraoral ferrule comprising:
an arch for preventing snoring and apnea; and
a ferrule configured to fit into a user's teeth and which incorporates said arch, wherein
the arch is U-shaped,
the arch protrudes from the anterior inner part of the ferrule and extensions od said arch are configured to go parallel to a bite plane to a point in the posterior final part of the ferrule,
the extensions are flexible,
the arch protrudes from the anterior inner part does so at the height of the incisors, the canines or the premolars, and
the extensions have a maximum inclination of 15° with respect to the bite plane.

16. The intraoral ferrule according to claim 15, wherein
the region has a curved shape.

17. The intraoral ferrule according to claim 15, wherein
the region has a geometric shape other than a curved shape.
